(19) United States
(12) Patent Application Publication
(10) Pub. No.: US 2007/0049933 A1
(43) Pub. Date: Mar. 1, 2007

(54) Multi-axial spinal pedicle screw

(76) Inventors: Sae Young Ahn, Pyeongtaig City (KR); Jae Kwan Lim, Pyeongtaig City (KR); Jong Myeon Choi, Suwon City (KR); Seo Kon Kim, Pyeongtaig City (KR); Hoon Kim, Pyeongtaig City (KR)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/292,783

(22) Filed: Dec. 2, 2005

(30) Foreign Application Priority Data
Aug. 30, 2005 (KR) 10-2005-0080299
Nov. 9, 2005 (KR) 10-2005-0107062

(51) Int. Cl. A61F 2/30

(52) U.S. Cl. 606/61

(57) ABSTRACT

Disclosed is a multi-axial spinal pedicle screw engaged with a spinal rod and inserted into and fixed to a pedicle. The multi-axial spinal pedicle screw includes: a screw rod in which a spherical articulation is integrally formed at an upper portion thereof, the screw rod being inserted into and fixed to the pedicle through a screw thread formed on the outer surface of a lower portion thereof; a spherical chuck having a plurality of claws, for surrounding and fixing the spherical articulation; a head section in which a receiving hole for receiving the spherical chuck fixing the spherical articulation is formed at a lower portion thereof so as to penetrate the lower portion, a rod receiving channel for receiving the spinal rod is opened upward, and an engagement screw root is formed on the inner surface of the rod receiving channel; and a tightening screw in which an engagement screw thread corresponding to the engagement screw root is formed on the outer surface thereof, the tightening screw being engaged with the head section through the rod receiving channel to fix the spinal rod.

### BACKGROUND OF THE INVENTION

1. Field of the Invention

The present invention relates to a spinal pedicle screw, and more particularly to a multi-axial spinal pedicle screw which can rotate a head section according to conditions during a surgery and includes a strong fixing force to prevent the release thereof after surgery.

2. Description of the Prior Art

A spine is a very complex system of a connection structure of bones which provide a body with a support portion and protect delicate spinal cords and nerve roots. A spine includes a series of stacked spinal columns and a pedicle of each spinal column includes the interior portion formed of relatively weak cavernous bones and the exterior portion formed of relatively strong cortical bones.

The treatment of a disorder related to a spine generally includes an indirect method in which physical treatments are performed and a direct method in which a separate fixation apparatus is mounted to a pedicle to correct and fix the spine. If the spinal disorder is slight, physical treatments can be performed, but if the disorder of cervical spines, thoracic vertebras, lumbar vertebras, sacrums, and intervertebral discs which forms the spine are severe, a treatment using a separate spinal fixation apparatus should be performed.

The spinal fixation technology using a separate spinal fixation apparatus as a direct treatment refers to a technology in which a spine is fixed by using an orthopedic rod extending substantially in parallel to the spine and conventionally designated as a spinal rod. The technology can be achieved by exposing the spine to the rear side and inserting the spinal pedicle screw into a pedicle of a spinal column.

Generally, two spinal pedicle screws are installed at each spinal column and function as a fixing point with respect to a spinal rod. Therefore, the spine is fixed so as to have a more advantageous shape by the arrangement of the spinal rod. A spinal pedicle screw includes a screw rod inserted into and fixed to the pedicle and a head section integrally engaged with the screw rod, and is classified into two categories according to the engagement manners of the screw rod and the head section.

One of them has a head section and a screw rod which are integrally formed and is generally designated as “a single-axis spinal pedicle screw”, and the other has a head section capable of rotating during the surgery, if necessary, and is generally designated as “a multi-axial spinal pedicle screw”.

In a multi-axial spinal pedicle screw, a screw rod in which a spherical articulation is formed at the upper portion thereof is inserted into a head section so as not to deviate from the lower portion of the head section and a spinal rod is pressed and fixed by a screw member, in order to constitute an axial shape of various types.

However, in the case of the conventional multi-axial spinal pedicle screw, the screw member engaged with the exterior can be loosened by strong external forces applied to surgical portions or by long time and continuous external forces caused by the activities of a patient. Further, the multi-axial spinal pedicle screw does not have the engagement force and resistance required for fixing a damaged spine that it has a difficulty in fixing the spine.

However, in order to satisfy the recent requirement for miniaturization, the diameter of a head section is prevented from being larger by screw-coupling a screw member, which engages a spinal rod and the head section, with the head section in which a screw root is formed on the inner surface thereof. But, since the above-mentioned structure cannot prevent the operation of the external forces, the screw member may be released over time or by the movements of the body and thus the spine cannot be securely fixed.

Further, in the case of the multi-axial spinal pedicle screw, since the fixing force is secured by inserting the spherical articulation of the upper portion of the screw rod into the head section and then using the contact friction with the inner surface of the head section, in order to engage the screw rod with the head section, it is difficult to fix the spine if a slide is generated between the spherical articulation and the inner surface of the head section, even in the case in which the screw is relatively stably engaged. Especially, in fixing the spherical articulation in a conventional manner, since the contact with the inner surface of the head section is almost a point contact, it is difficult to secure frictional fixing forces.

### SUMMARY OF THE INVENTION

Accordingly, the present invention has been made to solve the above-mentioned problems occurring in the prior art, and an object of the present invention is to provide a multi-axial spinal pedicle screw in which a head section and a tightening screw constitute a screw-coupling structure having tapered shapes in order to prevent the screws from being released even by strong external forces applied to surgical portions or by long time and continuous external forces, thereby stably fixing the spine.

It is another object of the present invention to provide a multi-axial spinal pedicle screw which is provided with at least one of a spherical chuck for surrounding and fixing the spherical articulation, a final engagement screw, and a biting plate, separately, so that a conventional structure in which the spherical articulation is supported only by point contact is converted to a structure in which the spherical articulation is fixed by surface contact and dual engagement structure, thereby maximizing the frictional engagement force between the spherical articulation and the inner surface of the head section.

It is another object of the present invention to provide a multi-axial spinal pedicle screw in which, since friction pads of a polymer are engaged with the contact portions such as the inner and outer peripheral surfaces of a spherical chuck, the inner and outer peripheral surfaces of a spherical articulation, the inner surface of a head section, and the like or prominences and depressions are formed on the contact portions, the mutual frictional engagement force between the head section and the screw rod is maximized.

It is another object of the present invention to provide a multi-axial spinal pedicle screw in which, since the thickness of a screw thread formed on the outer peripheral surface of a screw rod is maximal at the upper portion thereof, the fixing force according to the progress of rotation during the insertion of the screw rod into the pedicle is maximized.

In order to accomplish these objects, there is provided a multi-axial spinal pedicle screw engaged with a spinal rod and inserted into and fixed to a pedicle, which includes: a screw rod in which a spherical articulation is integrally formed at an upper portion thereof, the screw rod being inserted into and fixed to the pedicle through a screw thread formed on the outer surface of a lower portion thereof; a spherical chuck having a plurality of claws, for surrounding and fixing the spherical articulation; a head section in which a receiving hole for receiving the spherical chuck fixing the spherical articulation is formed at a lower portion thereof so as to penetrate the lower portion, a rod receiving channel for receiving the spinal rod is opened upward, and an engagement screw root is formed on the inner surface of the rod receiving channel; and a tightening screw in which an engagement screw thread corresponding to the engagement screw root is formed on the outer surface thereof, the tightening screw being engaged with the head section through the rod receiving channel to fix the spinal rod.

It is preferable that the multi-axial spinal pedicle screw further includes: a biting plate engaged with the upper side of the spherical chuck so that the spinal rod is positioned thereon, for restricting the movement of the spherical chuck when the spinal rod presses the biting plate.

It is preferable that the engagement screw root has a tapered shape and an engraved surface becomes smaller as it goes toward the center of the head section.

It is preferable that the receiving hole is curved in such a manner that the cross-section thereof becomes smaller as it goes toward the lower side thereof.

It is preferable that a friction pad formed of a polymer is engaged on the inner peripheral surface of the receiving hole.

It is preferable that a friction pad formed of a polymer is engaged on at least one of the inner and outer surfaces of each claw of the spherical chuck.

It is preferable that a wrench recess of a predetermined shape is formed at the center portion of the upper surface of the spherical articulation.

It is preferable that a friction pad formed of a polymer is engaged on the outer peripheral surface of the spherical articulation.

It is preferable that prominences and depressions are formed on the outer peripheral surface of the spherical articulation.

It is preferable that the thickness of the screw thread formed on the outer surface of the screw rod becomes thicker as it goes toward the spherical articulation so that it is maximal at the upper portion screw rod.

It is preferable that a wrench recess of a predetermined shape is formed at the center portion of the upper surface of the tightening screw.

It is preferable that a friction pad formed of a polymer is engaged with the lower surface of the tightening screw.

It is preferable that a receiving recess is formed on the lower surface of the biting plate according to the curvature of the spherical articulation.

It is preferable that a friction pad formed of a polymer is engaged with the inner peripheral surface of the receiving recess.

In order to accomplish these objects, there is also provided a multi-axial spinal pedicle screw engaged with a spinal rod and inserted into and fixed to a pedicle, which includes: a head section in which a rod receiving channel for receiving the spinal rod is opened upward, an engagement screw root for fixing the spinal rod is formed on the inner surface of the rod receiving channel, and an engagement hole of a predetermined size is formed at the lower portion thereof so as to penetrate the lower portion; a screw rod in which a spherical articulation is integrally formed at an upper portion thereof and is inserted through the engagement hole, the screw rod being inserted into and fixed to the pedicle through a screw thread formed on the outer surface of a lower portion thereof; a final engagement screw receiving the spherical articulation and screw-coupled with the engagement hole, for preventing the deviation of the spherical articulation; and a tightening screw in which an engagement screw thread corresponding to the engagement screw root is formed on the outer surface thereof, the tightening screw being engaged with the head section through the rod receiving channel to fix the spinal rod.

It is preferable that the multi-axial spinal pedicle screw further includes: a biting plate engaged with the upper side of the spherical chuck so that the spinal rod is positioned thereon, for restricting the movement of the spherical chuck when the spinal rod presses the biting plate.

It is preferable that the engagement screw root has a tapered shape and an engraved surface becomes smaller as it goes toward the center of the head section.

It is preferable that the inner peripheral surface of the final engagement screw is curved in such a manner that the cross-section thereof becomes smaller as it goes toward the lower side thereof.

It is preferable that a friction pad formed of a polymer is engaged on the inner peripheral surface of the final engagement screw.

It is preferable that a wrench recess of a predetermined shape is formed at the center portion of the upper surface of the spherical articulation.

It is preferable that a friction pad formed of a polymer is engaged on the outer peripheral surface of the spherical articulation.

It is preferable that prominences and depressions are formed on the outer peripheral surface of the spherical articulation.

It is preferable that the thickness of the screw thread formed on the outer surface of the screw rod becomes thicker as it goes toward the spherical articulation so that it is maximal at the upper portion screw rod.

It is preferable that a wrench recess of a predetermined shape is formed at the center portion of the upper surface of the tightening screw.

It is preferable that a friction pad formed of a polymer is engaged with the lower surface of the tightening screw.

It is preferable that a receiving recess is formed on the lower surface of the biting plate according to the curvature of the spherical articulation.

It is preferable that a friction pad formed of a polymer is engaged with the inner peripheral surface of the receiving recess.

In order to accomplish these objects, there is also provided a multi-axial spinal pedicle screw engaged with a spinal rod and inserted into and fixed to a pedicle, which includes: a screw rod in which a spherical articulation is integrally formed at an upper portion thereof, the screw rod being inserted into and fixed to the pedicle through a screw thread formed on the outer surface of a lower portion thereof; a head section in which a receiving hole for receiving the spherical articulation is formed at a lower portion thereof so as to penetrate the lower portion, a rod receiving channel for receiving the spinal rod is opened upward, and an engagement screw root is formed on the inner surface of the rod receiving channel; and a tightening screw in which an engagement screw thread corresponding to the engagement screw root is formed on the outer surface thereof, the tightening screw being engaged with the head section through the rod receiving channel to fix the spinal rod.

It is preferable that the multi-axial spinal pedicle screw further includes: a biting plate engaged with the upper side of the spherical chuck so that the spinal rod is positioned thereon, for restricting the movement of the spherical chuck when the spinal rod presses the biting plate.

It is preferable that the engagement screw root has a tapered shape and an engraved surface becomes smaller as it goes toward the center of the head section.

It is preferable that the receiving hole is curved in such a manner that the cross-section thereof becomes smaller as it goes toward the lower side thereof.

It is preferable that a friction pad formed of a polymer is engaged on the inner peripheral surface of the receiving hole.

It is preferable that a wrench recess of a predetermined shape is formed at the center portion of the upper surface of the spherical articulation.

It is preferable that a friction pad formed of a polymer is engaged on the outer peripheral surface of the spherical articulation.

It is preferable that prominences and depressions are formed on the outer peripheral surface of the spherical articulation.

It is preferable that the thickness of the screw thread formed on the outer surface of the screw rod becomes thicker as it goes toward the spherical articulation so that it is maximal at the upper portion screw rod.

It is preferable that a wrench recess of a predetermined shape is formed at the center portion of the upper surface of the tightening screw.

It is preferable that a friction pad formed of a polymer is engaged with the lower surface of the tightening screw.

It is preferable that a receiving recess is formed on the lower surface of the biting plate according to the curvature of the spherical articulation.

It is preferable that a friction pad formed of a polymer is engaged with the inner peripheral surface of the receiving recess.

### BRIEF DESCRIPTION OF THE DRAWINGS

The above and other objects, features and advantages of the present invention will be more apparent from the following detailed description taken in conjunction with the accompanying drawings, in which:

FIGS. 1a and 1b are cross-sectional views for showing the engagement structure of a multi-axial spinal pedicle screw according to a preferred embodiment of the present invention,

FIG. 1a shows a state before the engagement of the spinal rod, and FIG. 1b shows a state after the engagement of the spinal rod;

FIG. 2 is an exploded view for showing the multi-axial spinal pedicle screw shown in FIG. 1;

FIG. 3 is a perspective view for showing the appearance of the head section of the multi-axial spinal pedicle screw according to a preferred embodiment of the present invention;

FIGS. 4a and 4b are views for showing a tightening screw of a multi-axial spinal pedicle screw according to a preferred embodiment of the present invention,

FIG. 4a is a perspective view of the tightening screw, and FIG. 4b is a partially cutaway perspective view for showing the shape of an engagement screw thread;

FIG. 5 is a perspective view for showing the appearance of a biting plate of the multi-axial spinal pedicle screw according to a preferred embodiment of the present invention;

FIG. 6 is a perspective view for showing the appearance of a spherical chuck of the multi-axial spinal pedicle screw according to a preferred embodiment of the present invention;

FIG. 7 is a view for showing the appearance and cross-sectional structure of the screw rod of the multi-axial spinal pedicle screw according to a preferred embodiment of the present invention;

FIG. 8 is a view for showing another example of the screw rod shown in FIG. 7;

FIGS. 9a and 9b are views for explaining the operational principle of the engagement structure of the spherical articulation, the spherical chuck, and the biting plate according to a preferred embodiment of the present invention,

FIG. 9a shows a state before the spherical chuck is pressed downward and FIG. 9b is a fixed state after the spherical chuck is pressed downward;

FIGS. 10a and 10b are views for showing a cross-sectional structure in which a multi-axial spinal pedicle screw according to another preferred embodiment of the present invention and a spinal rod are engaged with each other,

FIG. 10a shows a state before the engagement of the spinal rod and FIG. 10b shows a state after the engagement of the spinal rod;

FIG. 11 is an exploded view for showing the multi-axial spinal pedicle screw shown in FIG. 10;

FIG. 12 is a perspective view for showing the appearance of the head section of the multi-axial spinal pedicle screw according to another preferred embodiment of the present invention;

FIGS. 13a and 13b are views for explaining the operational principle of the engagement structure of the spherical articulation, the final engagement screw, and the biting plate according to another preferred embodiment of the present invention,

FIG. 13a shows a state before the spherical articulation is pressed downward and FIG. 13b shows a fixed state after the spherical articulation is pressed downward;

FIGS. 14a and 14b are cross-sectional views for showing the engagement structure of a multi-axial spinal pedicle screw according to another preferred embodiment of the present invention,

FIG. 14a shows a state before the engagement of the spinal rod, and FIG. 14b shows a state after the engagement of the spinal rod;

FIG. 15 is an exploded view for showing the multi-axial spinal pedicle screw shown in FIG. 14; and

FIGS. 16a and 16b are views for explaining the operational principle of the engagement structure of the spherical articulation and the biting plate according to another preferred embodiment of the present invention,

FIG. 16a shows a state before the spherical articulation is pressed downward and FIG. 16b shows a fixed state after the spherical articulation is pressed downward.

### DETAILED DESCRIPTION OF THE PREFERRED EMBODIMENTS

Hereinafter, preferred embodiments of the present invention will be described with reference to the accompanying drawings.

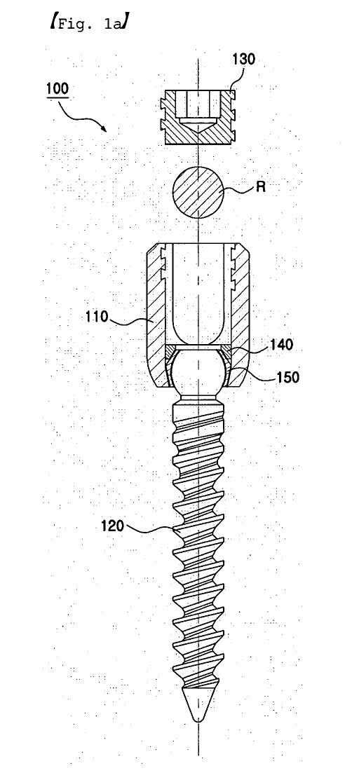

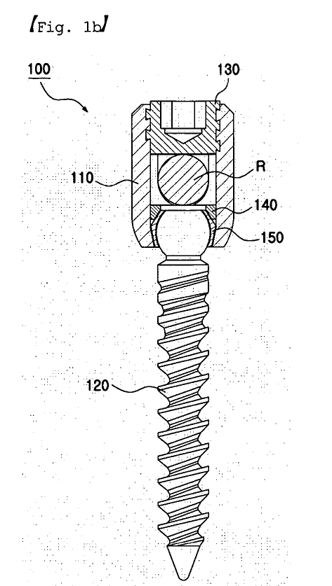

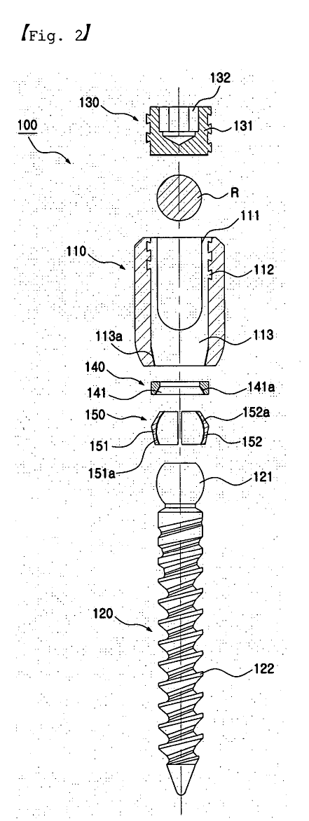

FIGS. 1a and 1b are cross-sectional views for showing the engagement structure of a multi-axial spinal pedicle screw according to a preferred embodiment of the present invention. FIG. 1a shows a state before the engagement of the spinal rod, and FIG. 1b shows a state after the engagement of the spinal rod. FIG. 2 is an exploded view for showing the multi-axial spinal pedicle screw shown in FIG. 1.

Referring to FIGS. 1 and 2, the multi-axial spinal pedicle screw 100 according to a preferred embodiment of the present invention includes a screw rod 120 in which a spherical articulation 121 is integrally formed at an upper portion thereof, a spherical chuck 150 having a plurality of claws 151 and 152 for surrounding and fixing the spherical articulation 121, a head section 110 in which a receiving hole 113 for receiving the spherical chuck 150, which fixes the spherical articulation 121, is formed so as to penetrate the lower portion thereof, a rod receiving channel 111 for receiving a spinal rod R is opened upward, and an engagement screw root 112 is formed on the inner surface of the rod receiving channel 111, a biting plate 140 engaged with the upper side of the spherical chuck 150 so that the spinal rod R can be positioned thereon, and a tightening screw 130 in which an engagement screw thread 131 corresponding to the engagement screw root 112 is formed on the outer surface thereof to engage the tightening screw 130 with the head section 110.

According to the multi-axial spinal pedicle screw 100, the spinal rod R is inserted into the head section 110 through the rod receiving channel 111 as shown in FIG. 1a and then the tightening screw 130 is screw-coupled with the head section 110 in order to have an engagement structure shown in FIG. 1b.

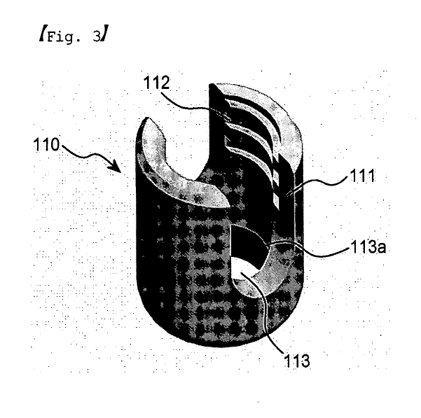

FIG. 3 is a perspective view for showing the appearance of the head section of the multi-axial spinal pedicle screw according to a preferred embodiment of the present invention.

Referring to FIGS. 2 and 3, the head section 110 is substantially cup-shaped and has a curved lower portion. The U-shaped rod receiving channel 111, which is opened upward, is formed on the side surface of the head section 110. A receiving hole 113 of a predetermined size is formed at the lower portion of the head section 110 so as to penetrate the lower portion of the head section 110.

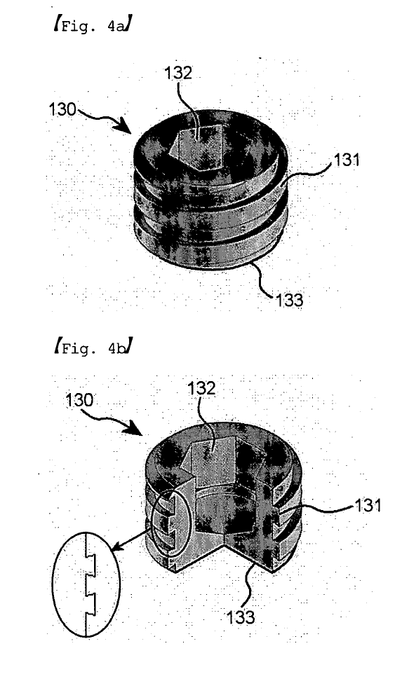

The interior interval of the rod receiving channel 111 is identical with the diameter of the spinal rod R engaged therewith so that the right and left movements of the engaged spinal rod R are restricted. The engagement screw root 112 specially molded so as to have a tapered shape (refer to FIG. 4b) is formed along the curved inner surface of the rod receiving channel 111.

Then, the central axis of the taper is perpendicular to the central axis of the head section 110, and the engraved surface becomes smaller as it goes toward the center of the head section 110. This is because, by using the above-mentioned structure, the actions of upward and downward external forces can be minimized and the engagement force can be maximized, when the tightening screw 130 having the engagement screw thread 131 is screw-coupled to the engagement screw root 112 of the head section 110.

The diameter of the receiving hole 113 is larger than the interval of the rod receiving channel 111 at the upper portion thereof, becomes smaller toward the lower side thereof with the receiving hole 113 curved, and is larger than the engagement cross-section of the spherical chuck 150, in order to stably receive the spherical chuck 150 surrounding and engaged with the spherical articulation 121, preventing the deviation of the spherical chuck 150.

On the other hand, it is preferable that a friction pad 113a formed of a polymer is integrally engaged on the inner peripheral surface of the receiving hole 113 to maximize the frictional engagement force between the receiving hole 113 and the spherical chuck 150. Further, according to a modified embodiment, prominences and depressions may be formed on the inner peripheral surface of the receiving hole 113 in order to obtain a similar effect.

FIGS. 4a and 4b are views for showing a tightening screw of a multi-axial spinal pedicle screw according to a preferred embodiment of the present invention. FIG. 4a is a perspective view of the tightening screw, and FIG. 4b is a partially cutaway perspective view for showing the shape of an engagement screw thread.

Referring to FIGS. 2 and 4, the engagement screw thread 131 corresponding to the engagement screw root 112 is formed on the outer surface of the tightening screw 130 so as to have a tapered shape, the cross-section of which becomes smaller as it goes toward the center of the head section 110. Preferably, hexagonal or octagonal wrench recess 132 is formed at the center portion of the tightening screw 130, so that the tightening screw 130 can be engaged with the head section 110 through the rod receiving channel 111 by means of a tightening device (not shown) such as a wrench, in order to fix the spinal rod R.

Then, it is preferable that, since the friction pad 133 of a polymer is engaged on the lower surface of the tightening screw 130, the frictional engagement force between the tightening screw 130 and the spinal rod R is maximized, thereby preventing the rotation and movement of the spinal rod R.

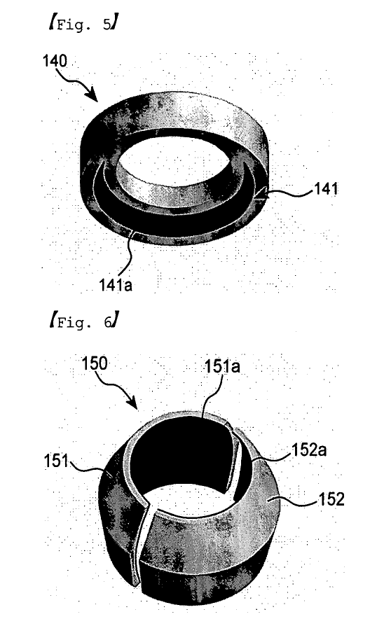

FIG. 5 is a perspective view for showing the appearance of a biting plate of the multi-axial spinal pedicle screw according to a preferred embodiment of the present invention.

Referring to FIGS. 2 and 5, the biting plate 140 is fixed between the receiving hole 113 and the spherical articulation 121. A receiving recess 141 is formed on the lower surface of the biting plate 140 according to the curvature of the spherical articulation 121 to restrict the movement of the spherical articulation 121 when the spinal rod R is pressed to be fixed.

Then, it is preferable that a friction pad 141a of a polymer is engaged on the inner peripheral surface of the receiving recess 141 so that the mutual frictional force between the biting plate 140 and the spherical chuck 150 can be improved when the biting plate 140 is engaged with the spherical chuck 150 by pressing the biting plate 140.

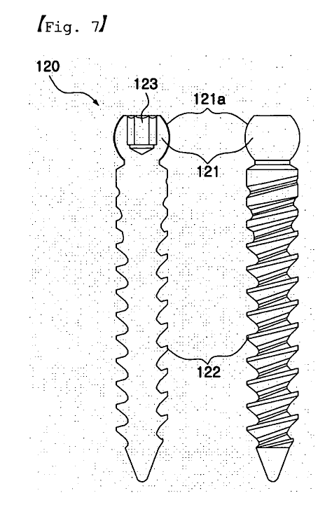

FIG. 6 is a perspective view for showing the appearance of the spherical chuck of the multi-axial spinal pedicle screw according to a preferred embodiment of the present invention.

Referring to FIGS. 2 and 6, the spherical chuck 150 includes a pair of claws 151 and 152 which are substantially semi-spherical. Each of the claws 151 and 152 is engaged with the spherical articulation 121, surrounding the side surface of the spherical articulation 121.

Then, it is preferable that friction pads 151a and 151b of a polymer are engaged on at least one of the inner and outer surfaces of each of the claws 151 and 152. Although the drawing shows the friction pads engaged only on the inner peripheral surfaces of the claws, the preferred embodiment of the present invention is not limited thereto. The above-mentioned structure applies strong frictional engagements force between the pair of claws 151 and 152 and the spherical articulation.

On the other hand, prominences and depressions may be formed on the inner and outer peripheral surfaces of the claws 151 and 152 in predetermined directions.

In addition, although the preferred embodiment of the present invention employs a pair of claws 151 and 152, three or four claws having a same shape may be used.

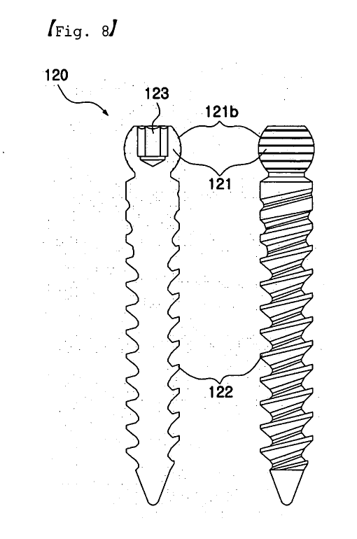

FIG. 7 is a view for showing the appearance and cross-sectional structure of the screw rod of the multi-axial spinal pedicle screw according to a preferred embodiment of the present invention and FIG. 8 is a view for showing another example of the screw rod shown in FIG. 7.

Referring to FIG. 7, the spherical articulation 121 is integrally formed at the upper portion of the screw rod 120 and a screw thread 122 is formed on the outer surface of the lower portion of the screw rod 120.

The upper surface of the spherical articulation 121 is cutaway to be flattened, and preferably a hexagonal or octagonal wrench recess 123 is formed at the center of the upper surface to be inserted into and fixed to a pedicle by means of a separate insertion device (not shown) such as a wrench.

A friction pad 121a of a polymer is integrally engaged on the outer peripheral surface of the spherical articulation 121 to maximize the mutual frictional force when the spherical chuck 150 and the biting plate 140 are pressed to be engaged with each other.

On the other hand, the lower body of the screw rod 120 on which the screw thread 122 is formed becomes smaller in the extending direction, and more particularly, the tip end of the lower portion has a conical shape to be easily inserted, and the diameter of the body of the screw rod 120 is maximal at the tip end of the upper portion thereof, and the thickness of the screw thread 122 becomes thicker as it goes toward the spherical articulation 121 so that it is maximal at the tip end of the upper portion thereof in order to maximize the fixing force according to the rotation when the screw rod 120 is inserted into the pedicle.

Referring to FIG. 8, prominences and depressions may be formed on the outer peripheral surface of the spherical articulation 121 in predetermined directions, in order to obtain a similar effect in which the frictional engagement force is maximized when the spherical chuck 150 and the biting plate 140 are engaged by pressing the biting plate 140. The shapes and directions of the prominences and depressions may be variously embodied and the scope of the present invention is not limited thereby.

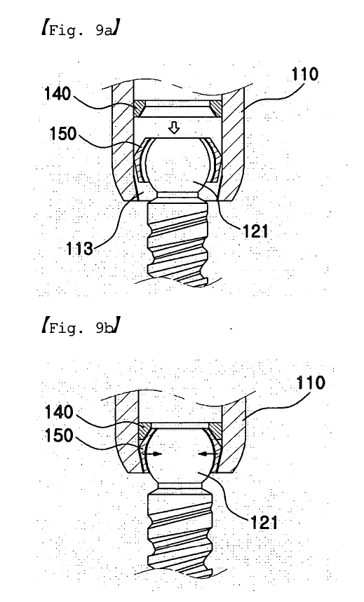

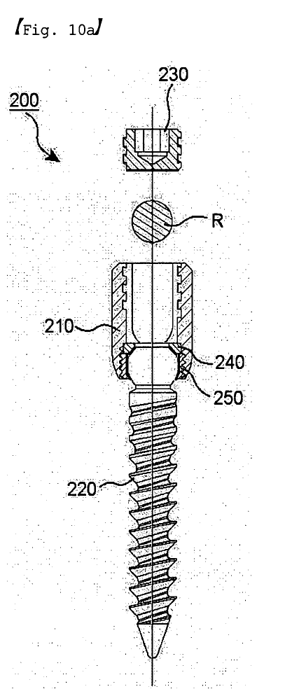

FIGS. 9a and 9b are views for explaining the operational principle of the engagement structure of the spherical articulation, the spherical chuck, and the biting plate according to a preferred embodiment of the present invention. FIG. 9a shows a state before the spherical chuck is pressed downward and FIG. 9b shows a fixed state after the spherical chuck is pressed downward.

Before the spinal rod (not shown) applies a pressing force, as shown in FIG. 9a, the spherical articulation 121 is separated from the spherical chuck 150 in the interior of the receiving hole 113 of the head section 110 so as to be freely moved, but, if the biting plate 140 pressed by the spinal rod applies a downward pressing force, as shown in FIG. 9b, the spherical articulation 121 is moved downward along the receiving hole 113 and thus a lateral pressing force is applied by means of the curvature of the receiving hole 113, so that the spherical articulation 121, the spherical chuck 150, and the biting plate 140 are integrally and strongly bitten.

Hereinafter, the engaging manners of the above-mentioned elements and the fixing manner of the spinal rod will be described in detail with reference to FIGS. 1 and 2.

As shown in FIGS. 1a and 2, the spherical chuck 150 surrounds the spherical articulation 121 is received in the receiving hole 113 of the head section 110 to determine a central axis of the screw rod 120 in various types according to surgical portions.

The multi-axial spinal pedicle screw 100 according to the present invention, which is engaged first, is inserted into and fixed to a pedicle through the wrench recess (not shown) formed on the upper surface of the spherical articulation 121 by a separate insertion device (not shown).

Thereafter, the biting plate 140 is inserted and positioned on the spherical chuck 150, and the spinal rod R is inserted on the upper side of the biting plate 140 along the rod receiving channel 111, and the tightening screw 130 is engaged on the upper side of the spinal rod R and is pressed and fixed by a separate tightening device (not shown).

The spinal rod R pressed by the tightening screw 130 presses the biting plate 140 making contact with the lower portion of the spinal rod R, and the spherical chuck 150 strongly bites the spherical articulation 121 by the contact with the inner surface of the receiving hole 113, thereby restricting the movement of the screw rod 120.

The multi-axial spinal pedicle screw 100 according to the present invention and the spinal rod R, which are finally engaged with each other, have an engagement structure as shown in FIG. 1b. The specially molded tapered shapes of the engagement screw root 112 of the head section 110 and the engagement screw thread 131 of the tightening screw 130 prevent them from being released by external forces applied to surgical portions. Further, since the spherical articulation 121 is fixed by surface contact and dual engagement structures of the spherical chuck 150 and the biting plate 140, the mutual engagement force between the spherical articulation 121 and the inner surface of the head section 110 can be maximized. Furthermore, the movement of the screw rod 120 after surgery is basically prevented by engaging friction pads of a polymer or forming prominences and depressions on the contact portions, thereby reinforcing the mutual frictional engagement forces.

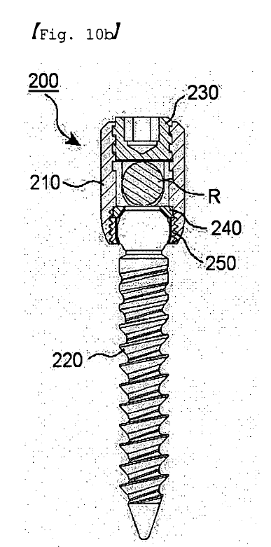

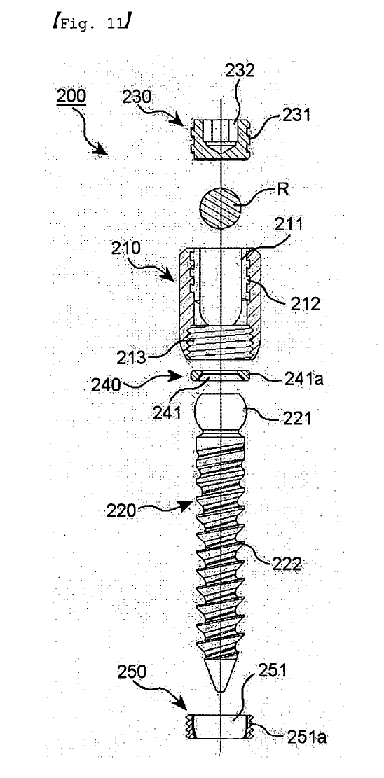

FIGS. 10a and 10b are views for showing a cross-sectional structure in which a multi-axial spinal pedicle screw according to another preferred embodiment of the present invention and a spinal rod are engaged with each other. FIG. 10a shows a state before the engagement of the spinal rod and FIG. 10b shows a state after the engagement of the spinal rod. FIG. 11 is an exploded view for showing the multi-axial spinal pedicle screw shown in FIG. 10. The elements of FIGS. 10 and 11, which are identical with the elements of FIGS. 1 and 2, will be given different reference numerals, but the detailed descriptions thereof will be omitted.

Referring to FIGS. 10 and 11, the multi-axial spinal pedicle screw 200 according to another preferred embodiment of the present invention includes a head section 210 in which a rod receiving channel 211 for receiving a spinal rod R is opened upward, an engagement screw root 212 for fixing the spinal rod R is formed on the inner surface of the rod receiving channel 211, and an engagement hole 213 is formed at the lower portion thereof so as to penetrate the lower portion thereof, a screw rod 220 in which a spherical articulation 221 is integrally formed at the upper portion thereof, a biting plate 240 inserted into the engagement hole 213 and engaged with the upper side of the spherical articulation 221, a final engagement screw receiving the spherical articulation 221 and screw-coupled with the engagement hole 213, and a tightening screw 230 in which an engagement screw thread 231 corresponding to the engagement screw root 212 is formed on the outer surface thereof to be screw-coupled with the head section 210.

According to the multi-axial spinal pedicle screw 200, the spinal rod R is inserted into the head section 210 through the rod receiving channel 211 as shown in FIG. 11a and then the tightening screw 230 is screw-coupled with the head section 210 in order to have an engagement structure shown in FIG. 11b.

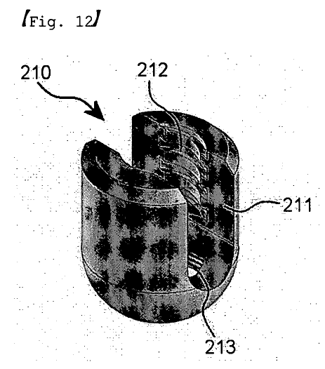

FIG. 12 is a perspective view for showing the appearance of the head section of the multi-axial spinal pedicle screw according to another preferred embodiment of the present invention.

Referring to FIGS. 11 and 12, the head section 210 is substantially cup-shaped and has a curved lower portion. The U-shaped rod receiving channel 211, which is opened upward, is formed on the side surface of the head section 210. A receiving hole 213 of a predetermined size is formed at the lower portion of the head section 210 so as to penetrate the lower portion of the head section 210.

The interior interval of the rod receiving channel 211 is identical with the diameter of the spinal rod R engaged therewith so that the right and left movements of the engaged spinal rod R are restricted. The engagement screw root 212 specially molded so as to have a tapered shape is formed along the curved inner surface of the rod receiving channel 211.

Then, the central axis of the taper is perpendicular to the central axis of the head section 210, and the engraved surface becomes smaller as it goes toward the center of the head section 210. This is because, by using the above-mentioned structure, the actions of upward and downward external forces can be minimized and the engagement force can be maximized, when the tightening screw 230 having the engagement screw thread 231 is screw-coupled to the engagement screw root 212 of the head section 210.

The engagement hole 213 penetrates the lower portion of the head section 210 so as to have a diameter larger than the interval of the rod receiving channel, and a catching portion for preventing the insertion of the biting plate 240 is formed at the border portion. Preferably, the catching portion is cut away so as to have a pair of wing. On the other hand, a screw root having a predetermined depth is formed on the outer surface of the engagement hole 213 in order to enable the engagement hole 213 to be screw-coupled with the final engagement screw.

Referring to FIGS. 10 and 11 again, the biting plate 240 is inserted into the engagement hole 213 of the head section 210 and engaged with the upper side of the spherical articulation 221. A receiving recess 241 is formed on the lower surface of the biting plate 240 along the curved surface of the spherical articulation 221 to restrict the movement of the spherical articulation 221 when the spinal rod R presses and fixes the biting plate 240.

Then, it is preferable that a friction pad 241a of a polymer is engaged with the inner peripheral surface of the receiving recess 241 in order to increase the mutual frictional force when the receiving recess 241 is engaged with the spherical articulation 221.

On the other hand, a penetration hole 251 for receiving the spherical articulation 221 is formed at the center of the final engagement screw 250. The lower portion of the inner peripheral surface of the penetration hole 251 I formed according to the curvature of the spherical articulation 221 to prevent the deviation of the spherical articulation 221, and a screw thread corresponding to the screw root of the engagement hole 213 is formed on the outer surface of the final engagement screw 250 to be adhered to and screw-coupled with the engagement hole 213.

Then, it is preferable that a friction pad 251a of a polymer is engaged with the inner peripheral surface of the penetration hole 251 to maximize the contact friction between the penetration hole 251 and the spherical articulation 221.

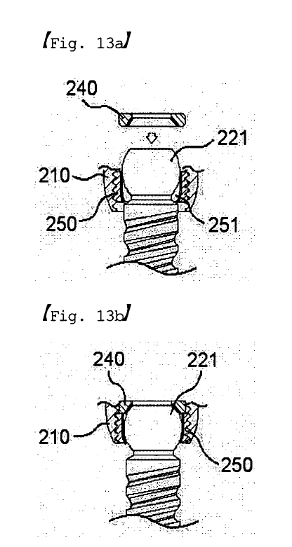

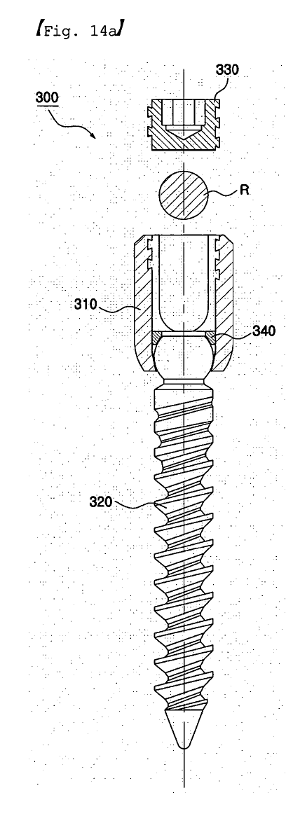

FIGS. 13a and 13b are views for explaining the operational principle of the engagement structure of the spherical articulation, the final engagement screw, and the biting plate according to another preferred embodiment of the present invention. FIG. 13a shows a state before the spherical articulation is pressed downward and FIG. 13b is a fixed state after the spherical articulation is pressed downward.

Before the spinal rod (not shown) applies a pressing force, as shown in FIG. 13a, the spherical articulation 221 can be freely moved in the interior of the final engagement screw 250 screw-coupled with the head section 210, but, if the biting plate 140 pressed by the spinal rod applies a downward pressing force, as shown in FIG. 13b, a lateral pressing force is applied by means of the curvature of the inner surface of the penetration hole 251, so that the spherical articulation 221, the final engagement screw 250, and the biting plate 240 are strongly bitten.

Hereinafter, the engaging manners of the above-mentioned elements and the fixing manner of the spinal rod will be described in detail with reference to FIGS. 10 and 11.

As shown in FIGS. 10a and 11, the biting plate 240 is inserted into the engagement hole 213 formed at the lower portion of the head section 210 and is fixed by the operation of the catching portion 213a, and the spherical articulation 221 is inserted into the engagement hole 213 to be adhered to and engaged with the receiving recess 241 of the biting plate 240, and the final engagement screw 250 penetrates through the screw rod 220 and is screw-coupled with the engagement hole 213 in order to prevent the deviation of the spherical articulation 221. Then, the spherical articulation 221 is freely moved and the central axis of the screw rod 220 is determined in various types according to the surgical portions.

The multi-axial spinal pedicle screw 200 according to the present invention, which is engaged first, is inserted into and fixed to a pedicle through the wrench recess (not shown) formed on the upper surface of the spherical articulation 121 by a separate insertion device (not shown).

Thereafter, the spinal rod R is inserted along the rod receiving channel 211, and the tightening screw 230 is screw-coupled with the head section 210 so that the spinal rod R is pressed and fixed by a separate tightening device (not shown).

The spinal rod R pressed by the tightening screw 230 presses the biting plate 240 making contact with the lower portion thereof and the spherical articulation 221 in order to maximizing the sealing/frictional engagement thereof, thereby restricting the movement of the screw rod 220.

The multi-axial spinal pedicle screw 200 according to the present invention and the spinal rod R, which are finally engaged with each other, have an engagement structure as shown in FIG. 10b. The specially molded tapered shapes of the engagement screw root 212 of the head section 210 and the screw thread 231 of the tightening screw 230 prevent them from being released by external forces applied to surgical portions. Further, the movement of the screw rod 120 after surgery is basically prevented by fixing the spherical articulation 221 by the surface contacts of the final engagement screw 250 and the biting plate 240 and engaging friction pads of a polymer or forming prominences and depressions on the contact portions to reinforce the mutual frictional engagement forces.

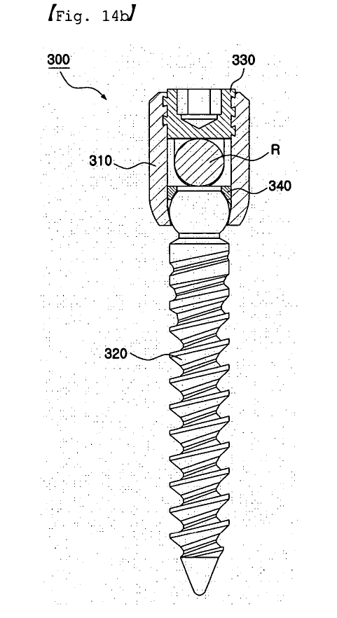

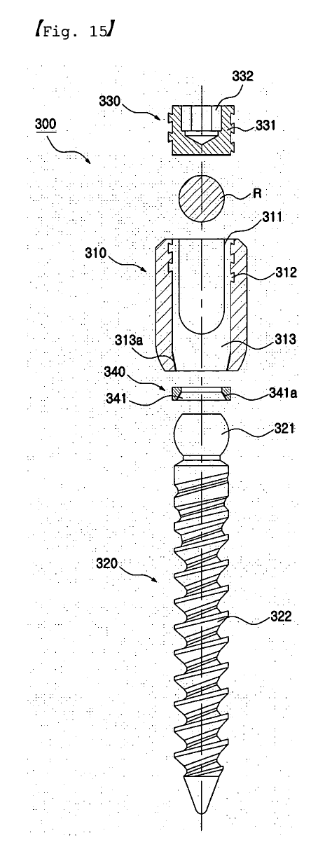

FIGS. 14a and 14b are cross-sectional views for showing the engagement structure of a multi-axial spinal pedicle screw according to a preferred embodiment of the present invention. FIG. 14a shows a state before the engagement of the spinal rod, and FIG. 14b shows a state after the engagement of the spinal rod. FIG. 15 is an exploded view for showing the multi-axial spinal pedicle screw shown in FIG. 14. The elements of FIGS. 14 and 14, which are identical with the elements of FIGS. 1 and 2, will be given different reference numerals, but the detailed descriptions thereof will be omitted.

Referring to FIGS. 14 and 15, the multi-axial spinal pedicle screw 300 according to another preferred embodiment of the present invention includes a screw rod 320 in which a spherical articulation 321 is integrally formed at an upper portion thereof, a head section 310 in which a receiving hole 313 for receiving the spherical articulation 321, is formed so as to penetrate the lower portion thereof, a rod receiving channel 311 for receiving a spinal rod R is opened upward, and an engagement screw root 312 is formed on the inner surface of the rod receiving channel 311, a biting plate 340 engaged with the upper side of the spherical articulation 321 so that the spinal rod R can be positioned thereon, and a tightening screw 330 in which an engagement screw thread 331 corresponding to the engagement screw root 112 is formed on the outer surface thereof to engage the tightening screw 330 with the head section 310.

According to the multi-axial spinal pedicle screw 300, the spinal rod R is inserted into the head section 310 through the rod receiving channel 311 as shown in FIG. 14a and then the tightening screw 330 is screw-coupled with the head section 310 in order to have an engagement structure shown in FIG. 14b.

Referring to FIG. 15, the engagement screw root 312 specially molded so as to have a tapered shape (refer to FIG. 4b) is formed along the curved inner surface of the rod receiving channel 311. Then, the central axis of the taper is perpendicular to the central axis of the head section 310, and the engraved surface becomes smaller as it goes toward the center of the head section 310. This is because, by using the above-mentioned structure, the actions of upward and downward external forces can be minimized and the engagement force can be maximized, when the tightening screw 330 having the engagement screw thread 331 is screw-coupled to the engagement screw root 312 of the head section 310.

The diameter of the receiving hole 313 is larger than the interval of the rod receiving channel 311 at the upper portion thereof, becomes smaller toward the lower side thereof with the receiving hole 313 curved, and is smaller than the engagement cross-section of the spherical articulation 321, in order to stably receive the spherical articulation 321, preventing the deviation of the spherical articulation 321.

On the other hand, it is preferable that a friction pad 313a formed of a polymer is integrally engaged on the inner peripheral surface of the receiving hole 313 to maximize the frictional engagement force between the receiving hole 313 and the spherical articulation 321. Further, according to a modified embodiment, prominences and depressions may be formed on the inner peripheral surface of the receiving hole 313 in order to obtain a similar effect.

The biting plate 340 is fixed between the receiving hole 313 and the spherical articulation 321. A receiving recess 341 is formed on the lower surface of the biting plate 340 according to the curvature of the spherical articulation 321 to restrict the movement of the spherical articulation 321 when the spinal rod R is pressed to be fixed.

Then, it is preferable that a friction pad 341a of a polymer is engaged on the inner peripheral surface of the receiving recess 341 so that the mutual frictional force between the biting plate 340 and the spherical articulation 321 can be improved when the biting plate 340 is engaged with the spherical articulation 321 by pressing the biting plate 340.

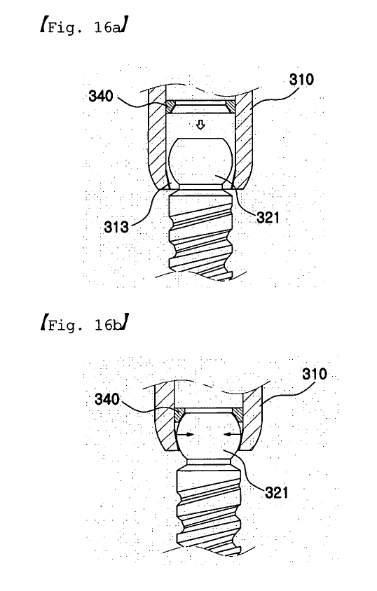

FIGS. 16a and 16b are views for explaining the operational principle of the engagement structure of the spherical articulation and the biting plate according to another preferred embodiment of the present invention. FIG. 16a shows a state before the spherical articulation is pressed downward and FIG. 16b shows a fixed state after the spherical articulation is pressed downward.

Before the spinal rod (not shown) applies a pressing force, as shown in FIG. 16a, the spherical articulation 321 is separated from the interior of the receiving hole 313 of the head section 310 so as to be freely moved, but, if the biting plate 340 pressed by the spinal rod applies a downward pressing force, as shown in FIG. 16b, the spherical articulation 321 is moved downward along the receiving hole 313 and thus a lateral pressing force is applied by means of the curvature of the receiving hole 313, so that the spherical articulation 321 and the biting plate 340 are integrally and strongly bitten.

Hereinafter, the engaging manners of the above-mentioned elements and the fixing manner of the spinal rod will be described in detail with reference to FIGS. 14 and 15.

As shown in FIGS. 14a and 15, the spherical articulation 321 is received in the receiving hole 313 of the head section 310 to determine a central axis of the screw rod 320 in various types according to surgical portions.

The multi-axial spinal pedicle screw 300 according to the present invention, which is engaged first, is inserted into and fixed to a pedicle through the wrench recess (not shown) formed on the upper surface of the spherical articulation 321 by a separate insertion device (not shown).

Thereafter, the biting plate 340 is inserted and positioned on the spherical articulation 321, and the spinal rod R is inserted on the upper side of the biting plate 340 along the rod receiving channel 311, and the tightening screw 330 is engaged on the upper side of the spinal rod R and is pressed and fixed by a separate tightening device (not shown).

The spinal rod R pressed by the tightening screw 330 presses the biting plate 340 making contact with the lower portion of the spinal rod R, and the spherical articulation 321 strongly contacts with the inner surface of the receiving hole 313, thereby restricting the movement of the screw rod 320.

The multi-axial spinal pedicle screw 300 according to the present invention and the spinal rod R, which are finally engaged with each other, have an engagement structure as shown in FIG. 14b. The specially molded tapered shapes of the engagement screw root 312 of the head section 310 and the engagement screw thread 331 of the tightening screw 330 prevent them from being released by external forces applied to surgical portions. Further, since the spherical articulation 321 is fixed by surface contact and dual engagement structures of the biting plate 340, the mutual engagement force between the spherical articulation 321 and the inner surface of the head section 310 can be maximized. Furthermore, the movement of the screw rod 320 after surgery is basically prevented by engaging friction pads of a polymer or forming prominences and depressions on the contact portions, thereby reinforcing the mutual frictional engagement forces.

According to the multi-axial spinal pedicle screw according to the present invention, the head section and the tightening screw constitute a screw-coupling structure having tapered shapes in order to prevent the screws from being released even by strong external forces applied to surgical portions or by long time and continuous external forces, thereby stably fixing the spine.

Further, the present invention is provided with at least one of the spherical chuck for surrounding and fixing the spherical articulation, the final engagement screw, and the biting plate, separately, so that the conventional structure in which the spherical articulation is supported only by point contact is converted to a structure in which the spherical articulation is fixed by surface contact and dual engagement structure, thereby maximizing the frictional engagement force between the spherical articulation and the inner surface of the head section.

Further, according to the present invention, since friction pads of a polymer are engaged with the contact portions such as the inner and outer peripheral surfaces of the spherical chuck, the inner and outer peripheral surfaces of the spherical articulation, the inner surface of the head section, and the like or prominences and depressions are formed on the contact portions, the mutual frictional engagement force between the head section and the screw rod is maximized.

Further, according to the present invention, since the thickness of the screw thread formed on the outer peripheral surface of the screw rod is maximal at the upper portion thereof, the fixing force according to the progress of rotation during the insertion of the screw rod into the pedicle is maximized.

Therefore, since the present invention can endure the strong external forces applied to the surgical portions of a patient after spine fixation surgeries, it can minimize side effects of the surgeries and help the recovery of the patient.

Although preferred embodiments of the present invention has been described for illustrative purposes, those skilled in the art will appreciate that various modifications, additions and substitutions are possible, without departing from the scope and spirit of the invention as disclosed in the accompanying claims.

What is claimed is:

1. A multi-axial spinal pedicle screw engaged with a spinal rod and inserted into and fixed to a pedicle, which comprises:
a screw rod in which a spherical articulation is integrally formed at an upper portion thereof, the screw rod being inserted into and fixed to the pedicle through a screw thread formed on the outer surface of a lower portion thereof;
a spherical chuck having a plurality of claws, for surrounding and fixing the spherical articulation;
a head section in which a receiving hole for receiving the spherical chuck fixing the spherical articulation is formed at a lower portion thereof so as to penetrate the lower portion, a rod receiving channel for receiving the spinal rod is opened upward, and an engagement screw root is formed on the inner surface of the rod receiving channel; and
a tightening screw in which an engagement screw thread corresponding to the engagement screw root is formed on the outer surface thereof, the tightening screw being engaged with the head section through the rod receiving channel to fix the spinal rod.

2. A multi-axial spinal pedicle screw according to claim 1, further comprising:
a biting plate engaged with the upper side of the spherical chuck so that the spinal rod is positioned thereon, for restricting the movement of the spherical chuck when the spinal rod presses the biting plate.

3. A multi-axial spinal pedicle screw according to claim 1, wherein the engagement screw root has a tapered shape and an engraved surface becomes smaller as it goes toward the center of the head section.

4. A multi-axial spinal pedicle screw according to claim 1, wherein the receiving hole is curved in such a manner that the cross-section thereof becomes smaller as it goes toward the lower side thereof.

5. A multi-axial spinal pedicle screw according to claim 4, wherein a friction pad formed of a polymer is engaged on the inner peripheral surface of the receiving hole.

6. A multi-axial spinal pedicle screw according to claim 1, wherein a friction pad formed of a polymer is engaged on at least one of the inner and outer surfaces of each claw of the spherical chuck.

7. A multi-axial spinal pedicle screw according to claim 1, wherein a wrench recess of a predetermined shape is formed at the center portion of the upper surface of the spherical articulation.

8. A multi-axial spinal pedicle screw according to claim 1, wherein a friction pad formed of a polymer is engaged on the outer peripheral surface of the spherical articulation.

9. A multi-axial spinal pedicle screw according to claim 1, wherein prominences and depressions are formed on the outer peripheral surface of the spherical articulation.

10. A multi-axial spinal pedicle screw according to claim 1, wherein the thickness of the screw thread formed on the outer surface of the screw rod becomes thicker as it goes toward the spherical articulation so that it is maximal at the upper portion screw rod.

11. A multi-axial spinal pedicle screw according to claim 1, wherein a wrench recess of a predetermined shape is formed at the center portion of the upper surface of the tightening screw.

12. A multi-axial spinal pedicle screw according to claim 1, wherein a friction pad formed of a polymer is engaged with the lower surface of the tightening screw.

13. A multi-axial spinal pedicle screw according to claim 2, wherein a receiving recess is formed on the lower surface of the biting plate according to the curvature of the spherical articulation.

14. A multi-axial spinal pedicle screw according to claim 13, wherein a friction pad formed of a polymer is engaged with the inner peripheral surface of the receiving recess.

15. A multi-axial spinal pedicle screw engaged with a spinal rod and inserted into and fixed to a pedicle, which comprises:
a head section in which a rod receiving channel for receiving the spinal rod is opened upward, an engagement screw root for fixing the spinal rod is formed on the inner surface of the rod receiving channel, and an engagement hole of a predetermined size is formed at the lower portion thereof so as to penetrate the lower portion;
a screw rod in which a spherical articulation is integrally formed at an upper portion thereof and is inserted through the engagement hole, the screw rod being inserted into and fixed to the pedicle through a screw thread formed on the outer surface of a lower portion thereof;
a final engagement screw receiving the spherical articulation and screw-coupled with the engagement hole, for preventing the deviation of the spherical articulation; and
a tightening screw in which an engagement screw thread corresponding to the engagement screw root is formed on the outer surface thereof, the tightening screw being engaged with the head section through the rod receiving channel to fix the spinal rod.

16. A multi-axial spinal pedicle screw according to claim 15, further comprising:
a biting plate engaged with the upper side of the spherical chuck so that the spinal rod is positioned thereon, for restricting the movement of the spherical chuck when the spinal rod presses the biting plate.

17. A multi-axial spinal pedicle screw according to claim 15, wherein the engagement screw root has a tapered shape and an engraved surface becomes smaller as it goes toward the center of the head section.

18. A multi-axial spinal pedicle screw according to claim 15, wherein the inner peripheral surface of the final engagement screw is curved in such a manner that the cross-section thereof becomes smaller as it goes toward the lower side thereof.

19. A multi-axial spinal pedicle screw according to claim 18, wherein a friction pad formed of a polymer is engaged on the inner peripheral surface of the final engagement screw.

20. A multi-axial spinal pedicle screw according to claim 15, wherein a wrench recess of a predetermined shape is formed at the center portion of the upper surface of the spherical articulation.

21. A multi-axial spinal pedicle screw according to claim 15, wherein a friction pad formed of a polymer is engaged on the outer peripheral surface of the spherical articulation.

22. A multi-axial spinal pedicle screw according to claim 15, wherein prominences and depressions are formed on the outer peripheral surface of the spherical articulation.

23. A multi-axial spinal pedicle screw according to claim 15, wherein the thickness of the screw thread formed on the outer surface of the screw rod becomes thicker as it goes toward the spherical articulation so that it is maximal at the upper portion screw rod.

24. A multi-axial spinal pedicle screw according to claim 15, wherein a wrench recess of a predetermined shape is formed at the center portion of the upper surface of the tightening screw.

25. A multi-axial spinal pedicle screw according to claim 15, wherein a friction pad formed of a polymer is engaged with the lower surface of the tightening screw.

26. A multi-axial spinal pedicle screw according to claim 16, wherein a receiving recess is formed on the lower surface of the biting plate according to the curvature of the spherical articulation.

27. A multi-axial spinal pedicle screw according to claim 26, wherein a friction pad formed of a polymer is engaged with the inner peripheral surface of the receiving recess.

28. A multi-axial spinal pedicle screw engaged with a spinal rod and inserted into and fixed to a pedicle, which comprises:
a screw rod in which a spherical articulation is integrally formed at an upper portion thereof, the screw rod being inserted into and fixed to the pedicle through a screw thread formed on the outer surface of a lower portion thereof;
a head section in which a receiving hole for receiving the spherical articulation is formed at a lower portion thereof so as to penetrate the lower portion, a rod receiving channel for receiving the spinal rod is opened upward, and an engagement screw root is formed on the inner surface of the rod receiving channel; and
a tightening screw in which an engagement screw thread corresponding to the engagement screw root is formed on the outer surface thereof, the tightening screw being engaged with the head section through the rod receiving channel to fix the spinal rod.

29. A multi-axial spinal pedicle screw according to claim 28, further comprising:
a biting plate engaged with the upper side of the spherical articulation so that the spinal rod is positioned thereon, for restricting the movement of the spherical articulation when the spinal rod presses the biting plate.

30. A multi-axial spinal pedicle screw according to claim 28, wherein the engagement screw root has a tapered shape and an engraved surface becomes smaller as it goes toward the center of the head section.

31. A multi-axial spinal pedicle screw according to claim 28, wherein the receiving hole is curved in such a manner that the cross-section thereof becomes smaller as it goes toward the lower side thereof.

32. A multi-axial spinal pedicle screw according to claim 31, wherein a friction pad formed of a polymer is engaged on the inner peripheral surface of the receiving hole.

33. A multi-axial spinal pedicle screw according to claim 28, wherein a wrench recess of a predetermined shape is formed at the center portion of the upper surface of the spherical articulation.

34. A multi-axial spinal pedicle screw according to claim 28, wherein a friction pad formed of a polymer is engaged on the outer peripheral surface of the spherical articulation.

35. A multi-axial spinal pedicle screw according to claim 28, wherein prominences and depressions are formed on the outer peripheral surface of the spherical articulation.

36. A multi-axial spinal pedicle screw according to claim 28, wherein the thickness of the screw thread formed on the outer surface of the screw rod becomes thicker as it goes toward the spherical articulation so that it is maximal at the upper portion screw rod.

37. A multi-axial spinal pedicle screw according to claim 28, wherein a wrench recess of a predetermined shape is formed at the center portion of the upper surface of the tightening screw.

38. A multi-axial spinal pedicle screw according to claim 28, wherein a friction pad formed of a polymer is engaged with the lower surface of the tightening screw.

39. A multi-axial spinal pedicle screw according to claim 29, wherein a receiving recess is formed on the lower surface of the biting plate according to the curvature of the spherical articulation.

40. A multi-axial spinal pedicle screw according to claim 39, wherein a friction pad formed of a polymer is engaged with the inner peripheral surface of the receiving recess.
